(19) United States
(12) Patent Application Publication
(10) Pub. No.: US 2008/0027306 A1
(43) Pub. Date: Jan. 31, 2008

(54) SYSTEM AND METHOD FOR INITIATING A DIAGNOSTIC IMAGING SCAN

(75) Inventors: Sheila S Washburn, Brookfield, WI (US); Michael R Figueira, Waukesha, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/461,035

(22) Filed: Jul. 31, 2006

(51) Int. Cl. A61B 5/05

(52) U.S. Cl. 600/410

(57) ABSTRACT

A method for initiating a diagnostic imaging scan for a diagnostic imaging system includes detecting an event. The event may be an event that indicates that the operator of the diagnostic imaging system is at a predetermined location. In response to the event, the diagnostic imaging scan is automatically started. Alternatively, the event may initiate a predetermined time period. Then, upon expiration of the predetermined time period, the diagnostic imaging scan is automatically started.

### FIELD OF THE INVENTION

The present invention relates generally to diagnostic imaging systems and in particular, to a method for initiating a diagnostic imaging scan based on an event.

### BACKGROUND OF THE INVENTION

Medical scanners and medical imaging machines may be used in various fields such as medical diagnostics. These scanners and medical imaging devices may utilize, for example, electromagnetic radiation, x-rays and sonic waves to produce images of a subject of interest, such as a patient, that are viewed by doctors for the diagnosis and care of the patient. A wide variety of diagnostic imaging systems have been developed such as, for example, x-ray systems, computer tomography (CT) systems and magnetic resonance imaging (MRI) systems. X-ray systems are useful for producing images of, for example, the chest, bones, joints and abdomen. CT systems are useful for producing images of, for example, organs, bones and other tissues. Conventional CT systems include an x-ray source and an x-ray detector, for example, mounted on a rotating gantry. The x-ray source and x-ray detector are rotated around the subject to capture image signals and a computer is used to create cross-sectional images of inside the body based on the image signals.

Magnetic resonance imaging (MRI) systems are useful for producing images of, for example, a wide range of soft tissues. MRI scanners typically use a computer to create images based on the operation of a magnet, a gradient coil assembly, and a radio frequency coil(s). The magnet creates a uniform main magnetic field that makes nuclei responsive to radio frequency excitation. The gradient coil assembly imposes a series of pulsed, spatial-gradient magnetic fields upon the main magnetic field to give each point in the volume a spatial identity corresponding to its unique set of magnetic fields during the imaging pulse sequence. The radio frequency coil creates an excitation frequency pulse that temporarily creates an oscillating transverse magnetization that is detected by the radio frequency coil and used by the computer to create the image. An image may be created using one of many known reconstruction techniques.

Various portions of a diagnostic imaging system, such as an imaging assembly and an operator console, may be located in separate rooms, such as a scan room and a control room, respectively. For example, in an MRI system typically the magnet assembly (including the magnet, gradient coil assembly, radio frequency coil, etc.) and patient table are enclosed in a scan room. The computer system and an operator console are located in a room adjacent to the scan room. For an MRI scan, typically an operator or technologist utilizes the operator console to, for example, enter patient information, enter scan parameters and load any scanning protocols. Then the technologist enters the scan room to set up the patient on the patient table. Once the patient is set up, the technologist then returns back to the operator console in the adjacent room to manually start the MRI scan. Even if the MRI scan is completely set up (e.g., all of the necessary information for at least the first series of the MRI scan has been entered), the technologist must return to the operator console to manually start the scan. During the time it takes the technologist to return to the operator console, the MRI scanner is not scanning and the technologist is not in communication with the patient.

It would be advantageous to provide a system and method to improve the efficiency, workflow and throughput of a diagnostic imaging system scan, such as an MRI scan, a CT scan, an X-ray, etc., in which the imaging assembly and controls are located in separate rooms. In particular, there is a need for a system and method for initiating a diagnostic imaging scan automatically based on the detection of an event. It would be desirable to automatically start a diagnostic imaging scan in response to an event that indicates that the operator has left the scan room.

### BRIEF DESCRIPTION OF THE INVENTION

In accordance with an embodiment, a method for initiating a diagnostic imaging scan for a diagnostic imaging system includes detecting an event that indicates an operator of the diagnostic imaging system is at a predetermined location, and, in response to the event, automatically starting the diagnostic imaging scan.

In accordance with another embodiment, a method for initiating a diagnostic imaging scan for a diagnostic imaging system includes detecting an event, in response to the event, initiating a predetermined time period and upon expiration of the predetermined time period, automatically starting the diagnostic imaging scan.

In accordance with another embodiment, a diagnostic imaging system includes an imaging assembly, and a computer system coupled to the imaging assembly and configured to control the operation of the imaging assembly and to automatically initiate a diagnostic imaging scan in response to an event that indicates an operator of the diagnostic imaging system is at a predetermined location.

### DESCRIPTION OF THE DRAWINGS

The invention will become more fully understood from the following detailed description, taken in conjunction with the accompanying drawings, in which:

FIG. 1 is a schematic block diagram of a magnetic resonance imaging system in accordance with an exemplary embodiment.

FIG. 2 is a flow chart illustrating a method for automatically initiating a diagnostic imaging scan based on an event in accordance with an embodiment.

FIGS. 3 and 4 are a flow chart illustrating a method for automatically initiating a diagnostic imaging scan based on an event in accordance with an alternative embodiment.

### DETAILED DESCRIPTION

The following description will refer to a magnetic resonance imaging (MRI) system, however, the systems and methods described herein may also be used in conjunction with other diagnostic imaging systems such as X-ray systems and CT systems. While a MRI system will be referred to throughout the discussion of various preferred embodiments, it should be understood that the embodiments of the systems and methods for initiating a diagnostic imaging scan are not limited to use with a MRI system.

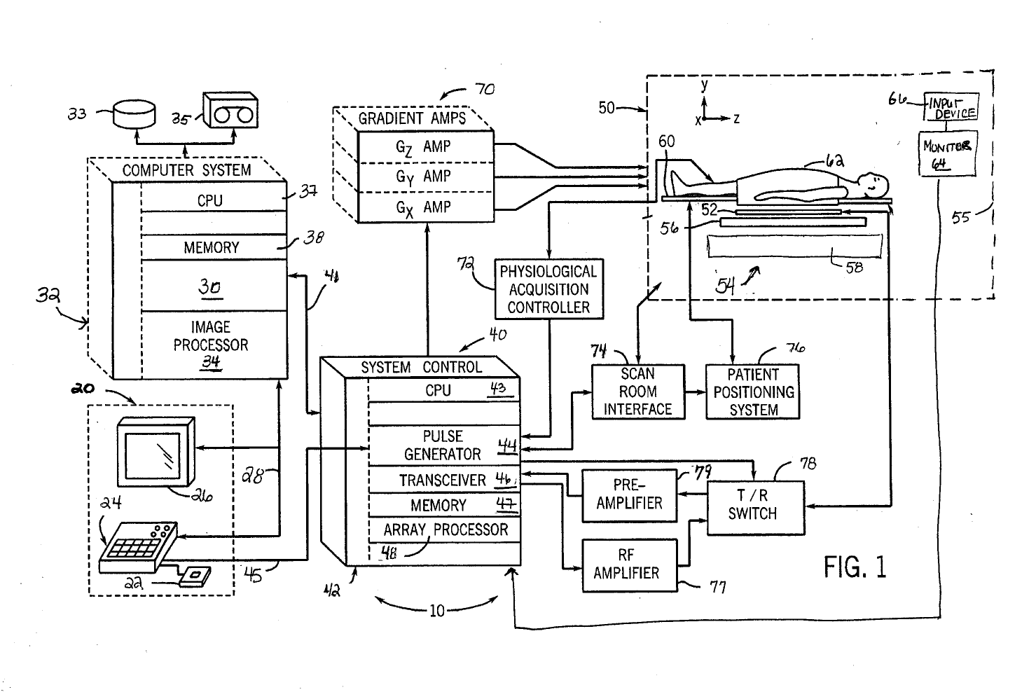

FIG. 1 is a schematic block diagram of a magnetic resonance imaging (MRI) system in accordance with an exemplary embodiment. The operation of the MRI system 10 is controlled from an operator console 20 that includes an input device 22, a control panel 24 and a display 26. The operator console 20 is located in a room separate from a scan room 50. The scan room 50 is a separate room having partition members 55 that enclose the scan room 50. A door (not shown) may be provided for access to the scan room 50. A patient table 60 and a magnet assembly 54 are located in the scan room 50. Accordingly, scan room 50 separates a patient 62, the patient table 60 and magnet assembly 54 from the operator console 20, a computer system 30 and a system control device 40 which are located in an adjacent room. In one embodiment, a monitor 64 and/or an input device 66 may be located in the scan room 50 to receive commands or operator input from an operator or technologist in the scan room 50. The input device 66 in the scan room 50 may include a mouse, joystick, keyboard, track ball, touch activated screen, light wand, voice control or any similar or equivalent input device.

The operator console 20 communicates through a link 28 with a separate computer system 30 that enables an operator to control the production and display of images on the screen 26. The computer system 30 includes a number of modules that communicate with each other through a backplane 32. These include an image processor module 34, a CPU module 37 and a memory module 38 such as a frame buffer for storing image data arrays. The computer system 30 is linked to a disk storage 33 and a tape drive 35 for storage of image data and programs. Computer system 30 also communicates with a separate system control 40 through a high speed serial link 41. The input device 22 may include a mouse, joystick, keyboard, track ball, touch activated screen, light wand, voice control or any similar or equivalent input device.

The system control 40 includes a set of modules connected together by a backplane 42. These include a CPU module 43 and a pulse generator module 44 that connects to the operator console 20 through a serial link 45. It is through link 45 that the system control 40 receives commands from the operator that indicate the scan sequence that is to be performed. The pulse generator module 44 operates the system components to carry out the desired scan sequence. Pulse generator module 44 produces data that indicates the timing, strength and shape of the RF pulses that are to be produced, and the timing and length of the data acquisition window. Pulse generator module 44 connects to a set of gradient amplifiers 70 to indicate the timing and shape of the gradient pulses to be produced during the scan. Pulse generator module 44 also receives patient data from a physiological acquisition controller 72 that receives signals from a number of different sensors connected to the patient, such as ECG signals from electrodes or respiratory signals from a bellows. In addition, pulse generator module 44 connects to a scan room interface circuit 74 that receives signals from various sensors associated with the condition of the patient and the magnet system. It is also through the scan room interface circuit 74 that a patient positioning system 76 receives commands to move the patient to the desired position for the scan.

The gradient waveforms produced by the pulse generator module 44 are applied to a gradient amplifier system 70 comprised of Gx, Gy and Gz amplifiers. Each gradient amplifier excites a corresponding gradient coil in a gradient coil assembly generally designated 56 to produce the magnetic field gradients used for position encoding acquired signals in the x-axis, y-axis and z-axis, respectively. The gradient coil assembly 56 forms part of a magnet assembly 54 that includes a magnet 58 and a whole-body RF coil 52. The magnet 58 could be a permanent magnet or an electromagnet. A patient or imaging subject 62 may be positioned within the magnet assembly 54, e.g., a bore formed by the magnet assembly 54. A transceiver module 46 in the system control 40 produces pulses that are amplified by an RF amplifier 77 and coupled to the RF coil 52 by a transmit/receive switch 78. The resulting signals radiated by the excited nuclei in the patient 62 may be sensed by the same RF coil 52 and coupled through the transmit/receive switch 78 to a preamplifier 79. The amplified NMR signals are demodulated, filtered and digitized in the receiver section of the transceiver 46. The transmit/receive switch 78 is controlled by a signal from the pulse generator module 44 to electrically connect the RF amplifier 77 to the coil 52 during the transmit mode and to connect the preamplifier 79 to the coil 52 during the receive mode. The transmit/receive switch 78 can also enable a separate RF coil (for example, a head coil or surface coil) to be used in either the transmit or receive mode.

The NMR signals picked up by the RF coil 52 are digitized by the transceiver module 44 and transferred to a memory module 47 in the system control 40. When the scan is completed and an entire array of data has been acquired in the memory module 47, an array processor 48 operates to Fourier transform the data into an array of image data. This image data is conveyed through the serial link 41 to the computer system 30 wherein it is stored in memory, such as the disk storage 33. In response to commands received from the operator console 20, the image data may be archived in long term storage, such as on the tape drive 35, or it may be further processed by the image processor 34 and conveyed to the operator console 20 and presented on the display 26.

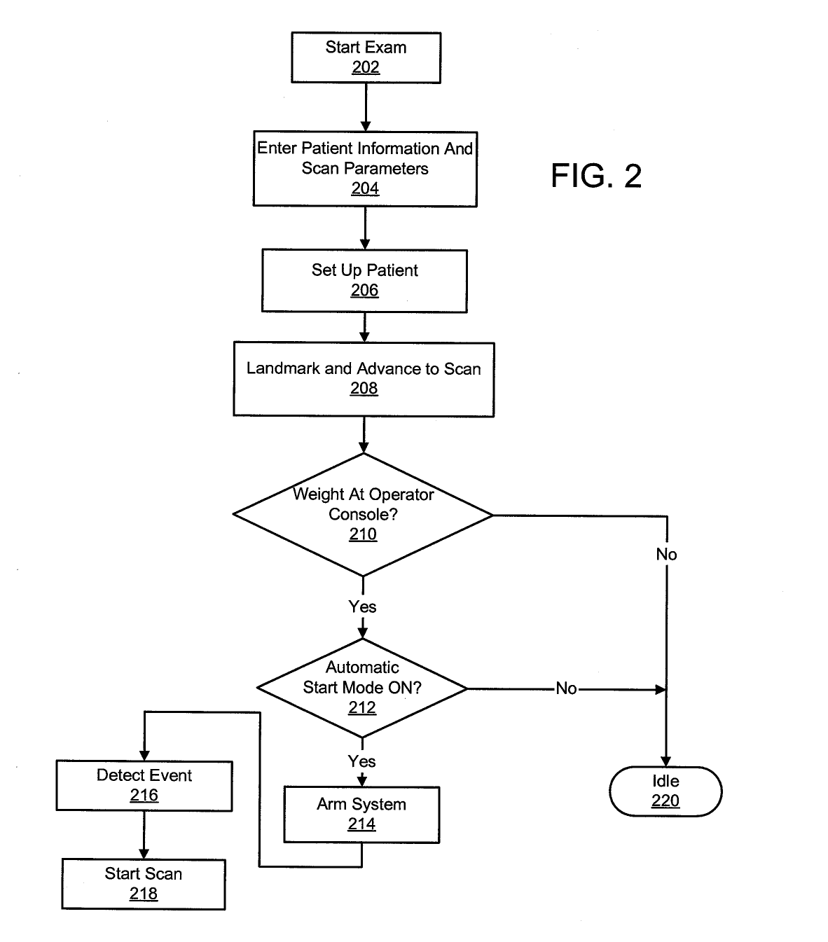

FIG. 2 is a flow chart illustrating a method for automatically initiating a diagnostic imaging scan based on an event in accordance with an embodiment. At block 202, the operator or technologist begins a diagnostic imaging exam, e.g., the MRI exam. For example, the operator may collect patient information or collect information regarding the procedure. The patient information, as well as parameters for the MRI scan, a protocol for the MRI scan, etc., may be entered at the operator console by the operator at block 204. As discussed above, with respect to FIG. 1, the operator console 20 and associated computer systems are located in a room adjacent to the scan room 50. Returning to FIG. 2, patient information may include, for example, a patient ID, name, birth date, sex, weight, age, radiologist, operator, reference, status, exam description and history. Scan parameters may include, for example, field of view (FOV), slice thickness, slices per frame and slice spacing. In one embodiment, the required patient information and scan parameters for at least a first scan series for the MRI scan is entered. Typically, an MRI scan consists of a plurality of imaging sequences or series, each lasting a predetermined period of time. Each series has its own degree of contrast and shows a cross section of the body in one of several planes (right to left, front to back, upper to lower). Often, the first series of a scan is a localizer series. In one embodiment, the settings for an automatic start mode may be saved as part of a protocol for the MRI scan. Alternatively, the automatic start (or autostart) mode for a scan may be selected and configured using the operator console.

Once the patient information and scan parameters are entered, the operator enters the scan room and sets up the patient on the patient table at block 206. The set up of the patient may include assisting the patient with positioning on the patient table, placing ECG leads on the patient, placing respiratory bellows on the patient, etc. At block 208, the operator may provide landmark information and advance to scan using controls provided on the magnet assembly in the scan room. A landmark is typically an anatomical structure used as a point of origin in locating other anatomical structures or used in determining certain measurements. When the operator selects to advance to scan, the patient table moves to an appropriate position within the magnet assembly for the scan. At block 210, if the patient's weight has not been entered at the operator console, the scanner remains idle. Patient weight is required before beginning an MRI scan. If the patient weight has been entered at block 210, the process proceeds to block 212.

At block 212, if the automatic start mode or feature is not on, the scanner remains idle at block 220. If the automatic start mode is on at block 212, then the magnet assembly or system is armed at block 214. When the MRI system detects a predefined event at block 216, the scan starts automatically at block 218. Preferably, the detected event is an event that indicates when the operator is at a predetermined location, for example, when the operator leaves the scan room. Accordingly, the operator does not need to return to the operator console to initiate the scan. In one embodiment, at least the first series of the scan starts at block 218. If a subsequent series of the scan requires the operator to reenter the scan room before the series starts, the automatic start mode may be used to automatically initiate the subsequent series when the operator leaves the scan room.

In one embodiment, a detection device, such as a sensor or camera, may be used to detect when the operator leaves the scan room. A signal may be provided from the detection device to the MRI system computer systems (e.g., computer system 30 or system control 40, both shown in FIG. 1) to indicate the detection of the event. In one embodiment, a door sensor may be positioned on or in proximity to the scan room door. The door sensor may be configured to detect the closure of the scan room door, e.g., the door sensor may sense a change in the state of the scan room door from open to closed. A signal may be provided from the door sensor to a computer system of the MRI system to indicate the detection of the event, i.e., the closure of the scan room door. In response to the detection of the event, the scan starts automatically at block 218. In alternative embodiments, a motion sensor may be used to detect when the operator leaves the scan room, a sensor may be used to detect when the operator crosses the threshold of the scan room door, a camera in the scan room may be used to detect when the operator leaves the scan room. Alternatively, a remote control mobile device (e.g., a remote controller) may be used by the operator to send a signal (e.g., to a computer system of the MRI system) to initiate the scan when the operator leaves the scan room.

In another embodiment, the event at block 216 consists of an event followed by a predetermined time period or delay. Upon expiration of the predetermined time period or delay, the scan starts automatically. Preferably, the predetermined time period or delay is a length (e.g., a number of seconds) that allows for the time it takes the operator to leave the scan room. For example, the operator may select a physical control in the scan room, such as a hard key, switch or button on the magnet assembly, a foot switch, a button or menu item on a display, which initiates a predetermined time period. The physical control may provide a signal to a computer system (e.g., computer system 30 or system control 40, both shown in FIG. 1) of the MRI system to indicate detection of the event. In alternative embodiments, a microphone located in the scan room may be used to receive a voice command(s) (e.g., voice activation) that initiates the predetermined time delay or, for scans that include the use of contrast agents, the activation of the power injector for the contrast agent may initiate the predetermined delay. Upon expiration of the delay, the scan is automatically started at block 218.

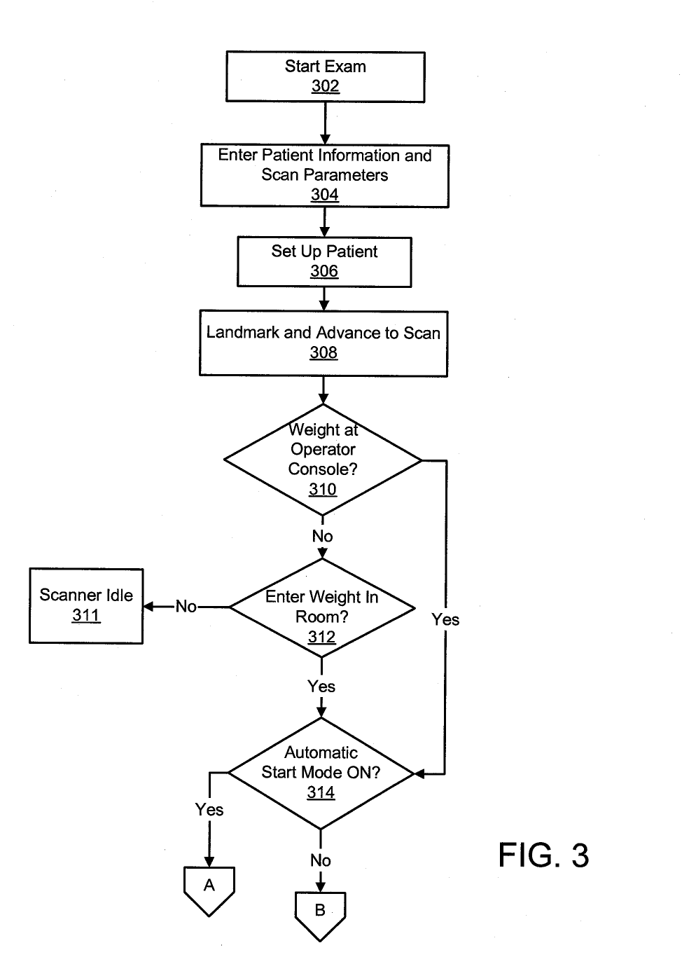

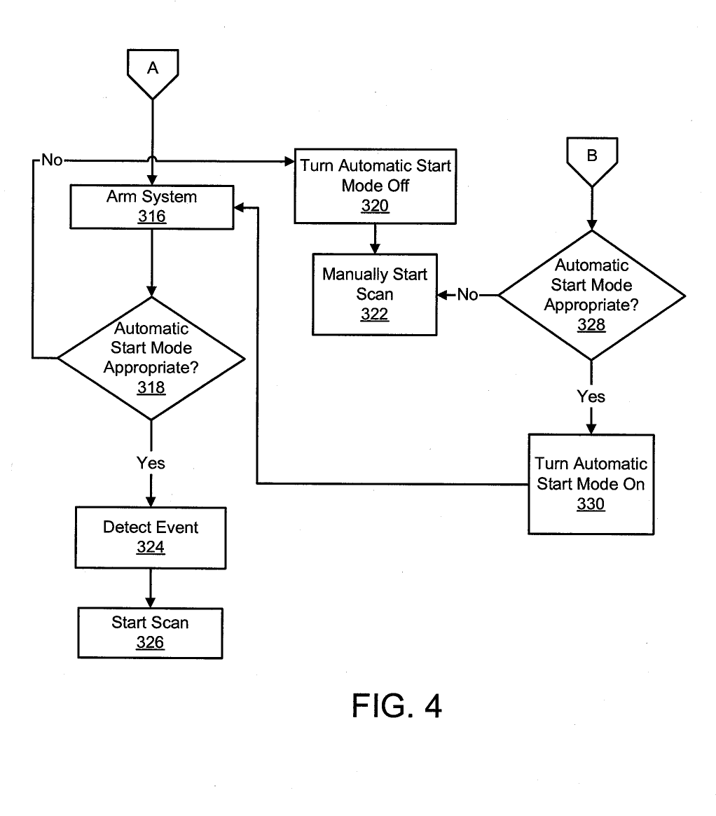

FIGS. 3 and 4 are a flow chart illustrating a method for automatically initiating diagnostic imaging scan based on an event in accordance with a alternative embodiment. In this embodiment, an operator may control the automatic start mode setting via controls in the scan room (e.g., a monitor 64 and/or an input device 66, shown in FIG. 1, in the scan room). In FIG. 3, at block 302, the operator or technologist begins the diagnostic imaging scan, e.g., the MRI exam. Patient information, as well as parameters for the MRI scan, a protocol for the MRI scan, etc., may be entered at the operator console by the operator at block 304. In one embodiment, the required patient information and scan parameters for at least a first scan series for the MRI scan is entered.

At block 306, the operator enters the scan room and sets up the patient on the patient table. The set up of the patient may include assisting the patient with positioning on the patient table, placing ECG leads on the patient, placing respiratory bellows on the patient, etc. At block 308, the operator may provide landmark information and advance to scan using controls provided on the magnet assembly in the scan room. At block 310, if the patient's weight has not been entered at the operator console, it is determined whether the patient's weight has been entered in the scan room at block 312, e.g., via a monitor and/or an input device or an input device on the magnet assembly. If the patient's weight has also not been entered in the scan room, the scanner remains idle at block 311. If the patient weight has been entered at the operator console at block 310 or in the scan room at block 312, the process proceeds to block 314.

At block 314, it is determined whether the automatic start mode or feature is on. If the automatic start mode is on, the magnet assembly or system is armed at block 316 in FIG. 4. Referring to FIG. 4, at block 318, it is determined whether the automatic start mode is appropriate for the particular scan. For example, the particular patient or procedure may not be compatible with using the automatic start mode. If the automatic start mode is appropriate at block 318, when the MRI system detects a predefined event at block 324, the scan starts automatically at block 326. As discussed above with respect to FIG. 2, preferably the detected event is an event that indicates when the operator leaves the scan room. In one embodiment, a detection device, such as a sensor or camera, may be used to detect when the operator leaves the scan room. Alternatively, the event may consist of an event followed by a predetermined time period or delay. The predetermined time period or delay may be a length of time that allows for the time it takes the operator to leave the scan room.

If the automatic start mode is not appropriate at block 318, the automatic start mode may be turned off at block 320 using controls in the scan room. For example, a monitor 64 and/or an input device 66 (both shown in FIG. 1) maybe used to provide input to disable the automatic start mode. The scan may then be manually started at block 322 using the operator console located outside of the scan room.

Returning to FIG. 3, if the automatic start mode is not on at block 314, it is determined whether the automatic start mode is appropriate for the particular scan at block 328 in FIG. 4. As shown in FIG. 4, if the automatic start mode is not appropriate, the automatic start mode is left off and the scan may then be manually started at block 322 using the operator console located outside of the scan room. If the automatic start mode is appropriate, the automatic start mode may be turned on at block 330. For example, a monitor 64 and/or an input device 66 (both shown in FIG. 1) may be used to provide input to enable the automatic start mode. The magnet assembly or system is armed at block 316. When the MRI system detects a predefined event at block 324, the scan starts automatically at block 326. As discussed above with respect to FIG. 2, preferably the detected event is an event that indicates when the operator leaves the scan room. In one embodiment, a detection device, such as a sensor or camera, may be used to detect when the operator leaves the scan room. Alternatively, the event may consist of an event followed by a predetermined time period or delay. The predetermined time period or delay may be a length of time that allows for the time it takes the operator to leave the scan room.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. The order and sequence of any process or method steps may be varied or re-sequenced according to alternative embodiments.

Many other changes and modifications may be made to the present invention without departing from the spirit thereof. The scope of these and other changes will become apparent from the appended claims.

We claim:

1. A method for initiating a diagnostic imaging scan for a diagnostic imaging system, the method comprising:
detecting an event that indicates an operator of the diagnostic imaging system is at a predetermined location; and
in response to the event, automatically starting the diagnostic imaging scan.

2. A method according to claim 1, wherein the diagnostic imaging scan is a magnetic resonance imaging scan and the diagnostic imaging systems is a magnetic resonance imaging system.

3. A method according to claim 1, wherein the diagnostic imaging system comprises an imaging assembly located in a scan room having a door.

4. A method according to claim 3, wherein the event is closure of the scan room door.

5. A method according to claim 4, wherein a sensor is used to detect closure of the scan room door.

6. A method according to claim 1, wherein a motion sensor is used to detect the event.

7. A method according to claim 3, wherein the event is determining when the operator crosses a threshold of the scan room door.

8. A method according to claim 1, wherein a camera is used to detect the event.

9. A method according to claim 1, wherein the event is receipt of a signal from a remote controller.

10. A method according to claim 2, wherein at least one setting related to the event is stored in a magnetic resonance imaging protocol.

11. A method according to claim 3, further comprising before detecting an event, enabling an automatic start mode via an input device in the scan room.

12. A method for initiating a diagnostic imaging scan for a diagnostic imaging system, the method comprising:
detecting an event;
in response to the event, initiating a predetermined time period; and
upon expiration of the predetermined time period, automatically starting the diagnostic imaging scan.

13. A method according to claim 12, wherein the diagnostic imaging scan is a magnetic resonance imaging scan and the diagnostic imaging system is a magnetic resonance imaging system.

14. A method according to claim 12, wherein the predetermined time period is a length of time that allows an operator of the diagnostic imaging system to reach a predetermined location.

15. A method according to claim 12, wherein the diagnostic system comprises an imaging assembly located in a scan room having a door.

16. A method according to claim 15, wherein the event is actuation of a physical control device in the scan room.

17. A method according to claim 15, wherein the event is receipt of at least one voice command via a microphone located in the scan room.

18. A method according to claim 15, wherein the event is activation of a contrast agent power injector located in the scan room.

19. A diagnostic imaging system comprising:
an imaging assembly; and
a computer system coupled to the imaging assembly, the computer system configured to control the operation of the imaging assembly and to automatically initiate a diagnostic imaging scan in response to an event that indicates an operator of the diagnostic imaging system is at a predetermined location.

20. A diagnostic imaging system according to claim 19, wherein the diagnostic imaging scan is a magnetic resonance imaging scan and the diagnostic imaging system is a magnetic resonance imaging system.

21. A diagnostic imaging system according to claim 19, wherein the imaging assembly is located in a scan room having a door.

22. A diagnostic imaging system according to claim 21, wherein the event is closure of the scan room door.

23. A diagnostic imaging system according to claim 20, wherein the computer system further configured to store a magnetic resonance imaging protocol that includes at least one setting related to the event.

24. A diagnostic imaging system according to claim 21, further comprising an input device located in the scan room and coupled to the imaging assembly, the input device configured to enable and disable an automatic start mode of the diagnostic imagining system.

25. A diagnostic imaging system according to claim 19, wherein the computer system is further configured to initiate a predetermined time period in response to detection of the event and to automatically initiate a diagnostic imaging scan upon expiration of the predetermined time period.
